(19) United States
(12) Patent Application Publication
(10) Pub. No.: US 2015/0297911 A1
(43) Pub. Date: Oct. 22, 2015

(54) MEDICAL EQUIPMENT

(71) Applicant: Panasonic Intellectual Property Management Co., Ltd., Osaka-shi, Osaka (JP)

(72) Inventor: Yusuke MORI, Osaka (JP)

(21) Appl. No.: 14/647,137

(22) PCT Filed: Nov. 27, 2013
(86) PCT No.: PCT/JP2013/006968

§ 371 Date: May 26, 2015

(30) Foreign Application Priority Data
Dec. 3, 2012 (JP) 2012-264049

(51) Int. Cl. A61N 5/06

(52) U.S. Cl. CPC A61N 5/06; A61N 2005/0626; A61N 2005/0632; A61N 2005/0655

(57) ABSTRACT

A medical device of the present invention includes input unit for inputting prescribing information prescribed by a doctor for an examined patient, treatment unit for carrying out a treatment operation based on the prescribing information, and driving unit mounted to the treatment unit and controlling treatment unit to be in an operable state. Input unit, treatment unit and driving unit are configured to be separable from each other. By providing treatment unit in which prescribing information has been input and driving unit separately, a treatment operation can be carried out at a desired place. Thus, a medical device, with which the patient him/herself is able to carry out the prescribed treatment operation at a predetermined place such as the patient's home, is achieved.

### TECHNICAL FIELD

The present invention relates to a medical device for treating a site in need of treatment, and in particular to a medical device assuming that an individual patient uses the medical device to achieve treatment at patient's home.

### BACKGROUND ART

Conventionally, various medical devices and management systems thereof, assuming that an individual patient carries out treatment at patient's home, have been put to practical use or have been proposed. For example, there is provided a medical equipment management device for managing prescription details prescribed by a doctor or a medical institution in a plurality of medical devices to be personally used by a patient, by using a portable storage medium. The medical equipment management device has a configuration capable of inputting various types of prescription details easily and without error. In addition, a medical device managed by medical equipment management device, and a management system of a medical institution have been proposed (see, for example, PTL 1).

Furthermore, a home medical device for exchanging various pieces of information including prescribing information between a hospital (a doctor) and a medical device via a postcard having a machine-readable code, has been proposed (see, for example, PTL 2).

Furthermore, a medical device which uses a computer system and is put on by a patient has been proposed. The computer system is equipped with a medical IC card by which a treatment instrument control program suitable for a symptom of an individual patient functions (see, for example, PTL 3). Thus, a treatment adjustment program suitable for a patient is rewritten by understanding a symptom of a patient located at a remote place (at home) in real time. As a result, rapid and accurate treatment can be carried out.

However, any of the above-mentioned medical devices assume that a medical device itself for actually treating a patient is set at a patient's home. Accordingly, types of treatment that can be carried out (number of acceptable prescriptions) are limited to functions of the medical device itself.

Usually, for example, in a case of drugs whose dosage thereof is changed, treatment is matched to an optimum prescription based on improvement of conditions of a site to be treated. Thus, also in medical devices, it is desired that prescription details are controlled to be contents optimum for a treated site which the medical device is applied.

For example, in the case of an automated dialysis device and an oxygen concentrator as known home medical devices, it is desirable that the oxygen concentration itself, the flow rate, and the like, are finely controlled depending on conditions of a patient. At this time, it is necessary to provide an oxygen concentration meter with a function of finely controlling the oxygen concentration itself or the like, that is to say, to provide the above-mentioned devices with a configuration capable of achieving various types of prescriptions, in advance. However, when the function of finely controlling is provided, patient's burdens, for example, an increase in size of the device shape and cost-up, have been increased.

### CITATION LIST

### Patent Literature

PTL 1: Japanese Patent Unexamined Publication No. 2010-231599

PTL 2: Japanese Patent Unexamined Publication No. H7-178060

PTL 3: Japanese Patent Unexamined Publication No. 2002-32477

### SUMMARY OF THE INVENTION

In order to solve the above-mentioned problems, a medical device of the present invention includes an input unit for inputting prescribing information prescribed by a doctor for an examined patient; a treatment unit for carrying out a treatment operation based on the prescribing information input by the input unit; and a driving unit mounted to the treatment unit and controlling the treatment unit to be in an operable state. The input unit, the treatment unit, and the driving unit are configured to be separable from each other. The treatment unit in which the prescribing information has been input and the driving unit are provided separately for enabling the treatment operation to be carried out at a desired place.

With this configuration, the treatment unit and the driving unit except for the input unit can be set at a desired place such as a patient's home. At this time, for example, when a prescription is required to be controlled in more detail, various types of the treatment units themselves are prepared, thus enabling selection corresponding to the detailed prescriptions. Thus, an entire shape of the medical device including the input unit, the selected treatment unit, and the driving unit can be prevented from being increased in size. As a result, home treatment can be achieved while a patient's burden due to cost-up is prevented. Specifically, similar to a case of treatment in which different number of drugs are prescribed depending on symptoms of a patient, various types of treatment units constituting the medical device are prepared in advance. Then, a doctor selects a treatment unit depending on the prescription and provides a patient with the selected treatment unit. Accordingly, it is not necessary to provide a medical device main body or a treatment unit itself with all of the configurations for achieving various types of treatment. As a result, various types of treatment can be achieved by using a medical device having a small size and being excellent in portability.

Furthermore, a prescription determined depending on the conditions of a patient by a doctor or a medical institution can be set to the treatment unit via the input unit, and the treatment unit can be operated via the driving unit at a patient's home. Thus, it is possible to achieve a medical device with which a patient himself/herself can carry out a treatment operation with the prescription determined for the patient under the management of the doctor or the medical institution.

### BRIEF DESCRIPTION OF THE DRAWINGS

FIG. 1 is a conceptual configuration diagram of a medical device in accordance with an exemplary embodiment of the present invention.

FIG. 2 is a schematic configuration perspective view of a phototherapy device as an example of the medical device.

FIG. 3 is a schematic diagram of an electric circuit of the phototherapy device as an example of the medical device.

### DESCRIPTION OF EMBODIMENTS

Hereinafter, an exemplary embodiment of the present invention is described with reference to drawings. Note here that the present invention is not intended to be limited to the exemplary embodiment.

### Exemplary Embodiment

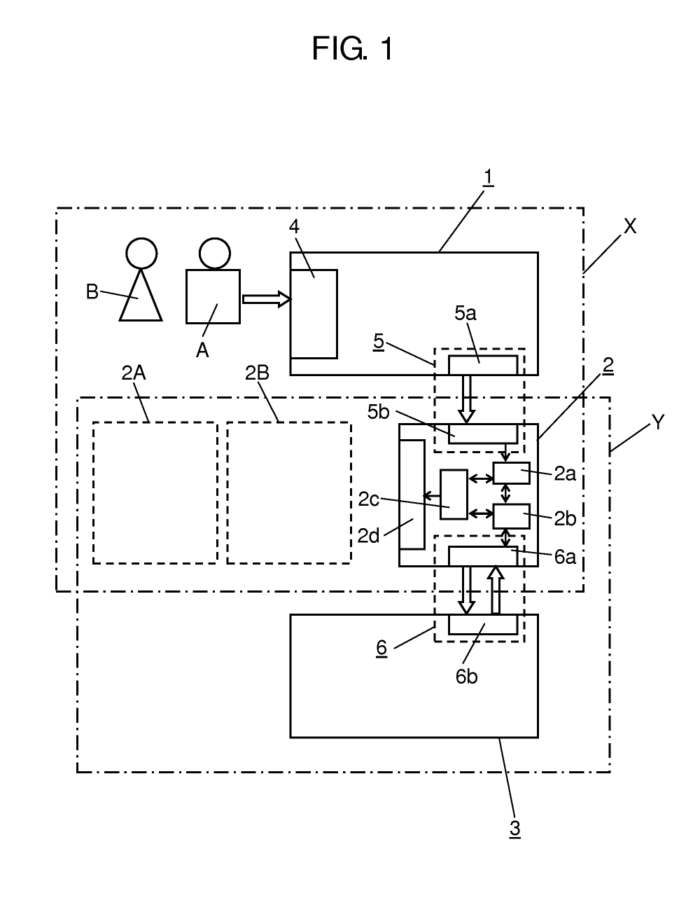

Hereinafter, a medical device in accordance with an exemplary embodiment of the present invention is described with reference to FIG. 1.

FIG. 1 is a conceptual configuration diagram of a medical device in accordance with an exemplary embodiment of the present invention.

As shown in FIG. 1, the medical device of this exemplary embodiment includes input unit 1, treatment unit 2, and driving unit 3, which are independently separable from each other.

In input unit 1, prescribing information prescribed by doctor A for examined patient B is input into treatment unit 2. Treatment unit 2 carries out a treatment operation based on the prescribing information input via input unit 1. Driving unit 3 controls treatment unit 2, which is mounted to driving unit 3, to be in an operable state.

In the medical device of this exemplary embodiment, medical institution X including doctor A inputs the prescribing information into treatment unit 2 by input unit 1, and prepares driving unit 3 for driving treatment unit 2. Then, the prepared treatment unit 2 and driving unit 3 are provided to patient B. Thus, patient B can mount treatment unit 2 to driving unit 3 by himself/herself at a desired place such as patient's home Y. As a result, patient B himself/herself can operate treatment unit 2 so as to carry out treatment based on the input prescribing information.

Furthermore, as shown in FIG. 1, input unit 1 includes at least input portion 4, and first coupling portion 5a constituting a part of first coupling mechanism unit 5. In input portion 4, for example, doctor A inputs prescribing information set depending on the examined patient B. Note here that input portion 4 can use a known input configuration such as a keyboard type, a dial type, and a push-button type.

Furthermore, the above-mentioned first coupling mechanism unit 5 includes first coupling portion 5a formed in input unit 1, and second coupling portion 5b formed in the below-mentioned treatment unit 2. Then, first coupling portion 5a of input unit 1 and second coupling portion 5b of treatment unit 2 are coupled to each other, so that prescribing information of patient B, which has been input via input portion 4 of input unit 1, is transmitted to treatment unit 2. Specifically, a known connector configuration including, for example, a plug and a socket, and a known wireless information transmission configuration can be appropriately used as first coupling portion 5a and second coupling portion 5b constituting first coupling mechanism unit 5.

Furthermore, as shown in FIG. 1, treatment unit 2 includes at least storage unit 2a, power supply unit 2b, control section 2c, treatment portion 2d, second coupling portion 5b constituting a part of first coupling mechanism unit 5, and third coupling portion 6a constituting a part of second coupling mechanism unit 6. Storage unit 2a stores prescribing information transmitted from input unit 1 via first coupling mechanism unit 5. Furthermore, storage unit 2a controls operations of power supply unit 2b, control section 2c, and the like, based on the stored prescribing information. Power supply unit 2b outputs energy necessary for treatment by treatment portion 2d in cooperation with the below-mentioned driving unit 3. Control section 2c controls operations of power supply unit 2b, treatment portion 2d, and the like. Treatment portion 2d carries out a treatment operation with respect to an affected part to be treated of patient B.

In addition, the above-mentioned second coupling mechanism unit 6 includes third coupling portion 6a formed in treatment unit 2, and fourth coupling portion 6b formed in driving unit 3. Then, by coupling third coupling portion 6a of treatment unit 2 and fourth coupling portion 6b of driving unit 3 to each other, the prescribing information, which is input by input portion 4 of input unit 1, of patient B is transmitted to driving unit 3 via treatment unit 2. Thus, control section 308 of driving unit 3 controls, for example, a voltage. Similarly, an operation output from driving unit 3, for example, energy necessary for treatment, is transmitted to power supply unit 2b of treatment unit 2. Specifically, a known connector configuration including, for example, configurations of a plug and a socket, a known non-contact energy transmission configuration, and the like, can be appropriately used as third coupling portion 6a and fourth coupling portion 6b constituting second coupling mechanism unit 6.

Furthermore, as shown in FIG. 1, driving unit 3 is coupled to treatment unit 2 via second coupling mechanism unit 6. Driving unit 3 controls power supply unit 2b of treatment unit 2 to be in a state capable of outputting energy necessary for treatment. At this time, when the above-mentioned power supply unit 2b is formed of, for example, a secondary battery, a capacitor, or the like, for accumulating energy necessary for treatment, driving unit 3 is configured as a mere power supply for supplying power supply unit 2b with energy. Then, power supply unit 2b of treatment unit 2 is coupled to driving unit 3 as a power supply, and thereby, it starts an operation of accumulating energy necessary for treatment. At this time, an operation of power supply unit 2b for outputting energy necessary for treatment or an operation of driving unit 3 as a power supply is controlled based on the prescribing information of patient B stored in storage unit 2a of treatment unit 2. Thus, a treatment operation by treatment unit 2 depending on the prescribing information of patient B is achieved.

That is to say, according to the configuration of the medical device of this exemplary embodiment shown in FIG. 1, in treatment unit 2, the operations of power supply unit 2b, control section 2c, and driving unit 3 are controlled based on the prescribing information prescribed by a doctor based on the examination of patient B, input by input unit 1 and stored in storage unit 2a. Thus, control section 2c of treatment unit 2 controls the operation of treatment portion 2d. Then, treatment portion 2d of treatment unit 2 carries out a treatment operation to the affected part of patient B based on the above-mentioned prescribing information.

At this time, patient B can prepare treatment unit 2 and driving unit 3, which are provided from medical institution X, at a predetermined place such as at home. Thus, patient B can undergo treatment at home based on the prescription by a doctor by using treatment unit 2. That is to say, patient B need not frequently go to medical institution X for undergoing treatment. As a result, a burden in terms of, for example, cost can be reduced.

Note here that third coupling portion 6a of treatment unit 2 and fourth coupling portion 6b of driving unit 3 need not be always coupled to each other by second coupling mechanism unit 6. In other words, when power supply unit 2b of treatment unit 2 has a configuration capable of accumulating and outputting energy necessary for treatment by the operation of driving unit 3 based on the prescribing information stored in storage unit 2a, treatment unit 2 and driving unit 3 need not always be coupled to each other. Thus, in a state in which coupling between treatment unit 2 and driving unit 3 via second coupling mechanism unit 6 is released, treatment by treatment unit 2 can be achieved by using energy accumulated in power supply unit 2b.

Furthermore, in this exemplary embodiment, after energy necessary for treatment is output from driving unit 3 to power supply unit 2b, and accumulated therein, driving unit 3 can be separated from treatment unit 2. Thus, a treatment operation by treatment portion 2d of treatment unit 2 can be carried out in places other than a place in which driving unit 3 is set. As a result, the degree of freedom in selecting treatment places can be extremely increased.

Furthermore, as shown in FIG. 1, treatment unit 2 belongs to both an area of medical institution X and an area of patient's home Y. That is to say, in the medical device of this exemplary embodiment, similar to general drugs, a doctor or medical institution X can provide patient B with treatment unit 2 in which prescribing information of patient B has been input. As a result, patient B himself/herself can use treatment unit 2 for treatment at patient's home Y, same as when a drug is taken.

Note here that as shown in FIG. 1, in addition to treatment unit 2, for example, a plurality of independent treatment units 2A and 2B having different treatment effects may be prepared for each prescription details. In this case, an operation state is controlled depending on the prescribing information of patient B in each of treatment units 2A and 2B. Thus, the treatment operation of patient B can be achieved while prescription details by a doctor or medical institution X are controlled to be more accurate contents.

Specifically, for example, three types of treatment units 2, 2A, and 2B are prepared for each prescription details of patient B. Then, each of treatment units 2, 2A, and 2B is configured so that a further plurality of operation states can be controlled. Thus, a doctor or medical institution X can select 3 types×(multiply) a plurality of treatment prescriptions as a whole. Consequently, as compared with a conventional case in which all of the many treatment prescriptions are achieved by a medical device main body, the shape of the medical device is not required to be larger. Thus, patient B need not bear the cost of configurations unnecessary for treatment prescription of the medical device. As a result, the medical device is advantageous for patient B in terms of setting space of the medical device and in terms of cost.

Furthermore, the medical device having the above-mentioned configuration would deal with the prescribing information of patient B as personal information. Accordingly, in this exemplary embodiment, it is preferable that first coupling mechanism unit 5 and second coupling mechanism unit 6 are provided with identifying functions such as a first identifying portion and a second identifying portion, which identify respective coupling counterparts. At this time, when, for example, the configurations of the first coupling portion and the second coupling portion have a connector configuration, the identifying functions can be achieved by known identifying technology, for example, a mechanical identifying portion in which a plug or a socket is made to have special shapes, an electrical identifying portion in which a signal is conferred after coupling, and the like.

Thus, treatment unit 2 operates only at a place where driving unit 3 is set. As a result, patient B can carry out treatment by himself/herself reliably by using identified treatment unit 2 based on the prescribing information input by a doctor or the like from input unit 1.

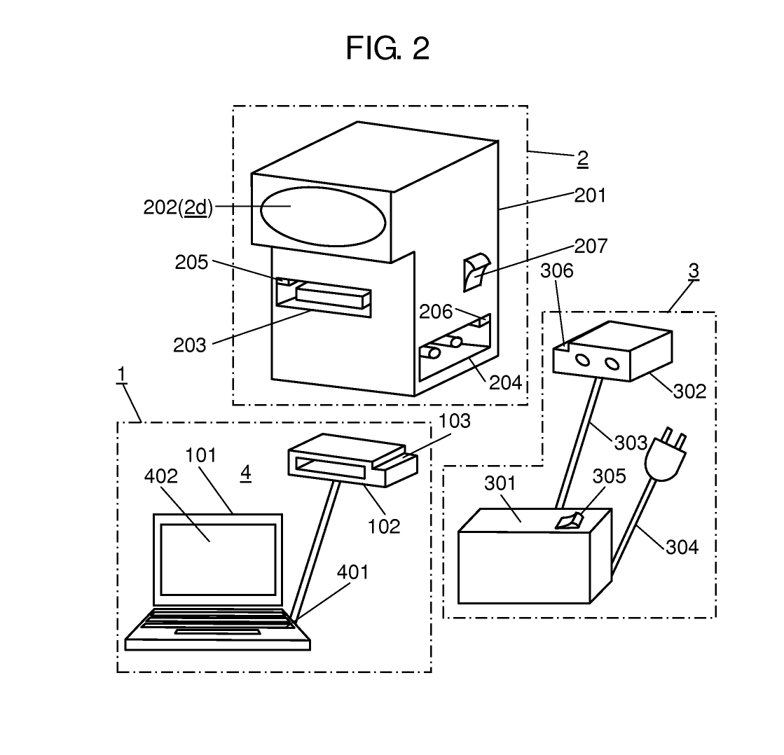

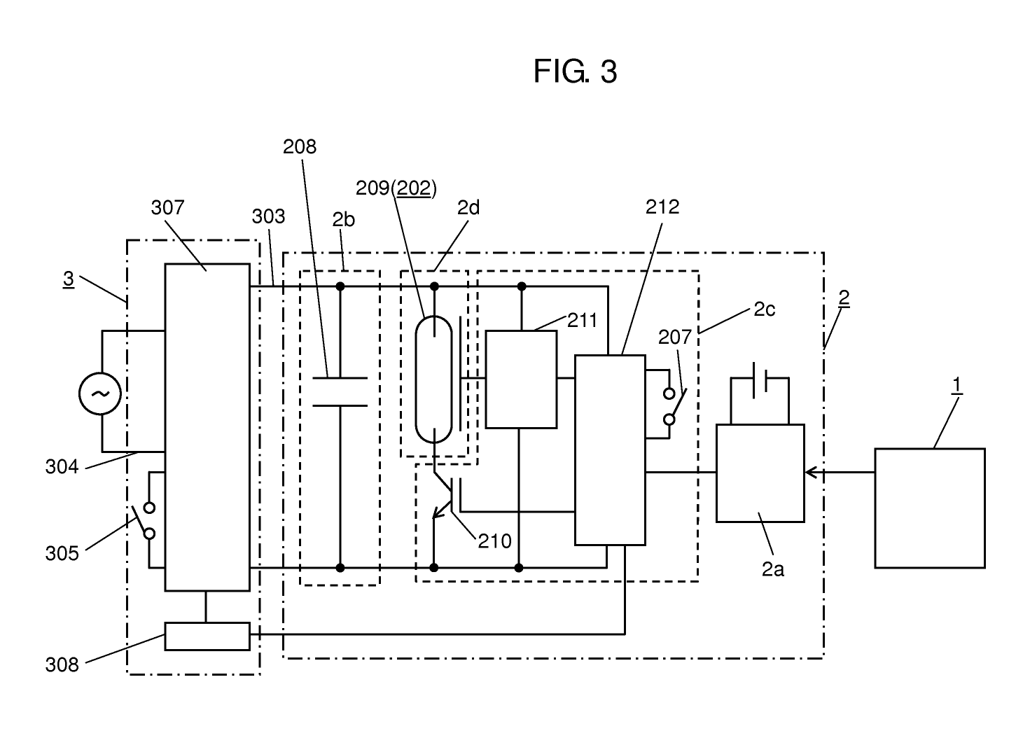

Hereinafter, the medical device of this exemplary embodiment is described with reference to FIGS. 2 and 3, taking a phototherapy device as an example.

Firstly, a configuration of the phototherapy device as the medical device of this exemplary embodiment is described with reference to FIG. 2.

FIG. 2 is a schematic configuration view of the phototherapy device as an example of the medical device.

As shown in FIG. 2, input unit 1 of the phototherapy device includes input portion 4, and first coupling portion 5a formed of, for example, plug 102. Input portion 4 is formed of a well-known information input portion provided to main body 101, and examples thereof include keyboard 401 and input screen 402, into which a doctor inputs prescribing information corresponding to an examined patient B. Plug 102 constitutes a part of first coupling mechanism unit 5, and is configured to input the prescribing information of patient B, which has been input from input portion 4, into treatment unit 2. Specifically, plug 102 is inserted into and set to socket 203 of treatment unit 2 constituting second coupling portion 5b that is a first part of first coupling mechanism unit 5 mentioned below. Thus, the prescribing information of patient B, which has been input from input portion 4, is input into treatment unit 2.

In this case, in this exemplary embodiment, first coupling mechanism unit 5 includes plug 102 as first coupling portion 5a and socket 203 as second coupling portion 5b.

Furthermore, in this exemplary embodiment, first different-shaped portion 103 constituting a first identifying portion is formed in a part of plug 102, and second different-shaped portion 205 constituting a second identifying portion that can be fitted into first different-shaped portion 103 is formed in a part of socket 203. Thus, input unit 1 and treatment unit 2 can identify each other. That is to say, when first different-shaped portion 103 of input unit 1 and second different-shaped portion 205 of treatment unit 2 are fitted to each other, treatment unit 2 into which prescribing information of patient B is input from input unit 1 is identified. As a result, input unit 1 and treatment unit 2 can be combined with each other reliably.

Furthermore, treatment unit 2 of the phototherapy device includes main body 201 provided with at least light-emitting portion 202, socket 203, socket 204, starting switch 207, and the like. Light-emitting portion 202 constitutes treatment portion 2d, and emits light so as to carry out a treatment operation for patient B. Socket 203 is coupled to plug 102 as first coupling portion 5a of input unit 1, and constitutes second coupling portion 5b that is a part of first coupling mechanism unit 5. Socket 204 is coupled to plug 302 that is the below-mentioned fourth coupling portion 6b of driving unit 3 and constitutes third coupling portion 6a that is a part of second coupling mechanism unit 6. Starting switch 207 starts a light-emitting operation of light-emitting portion 202. Needless to say, a shape of main body 201 of treatment unit 2 is formed into a shape assuming that it is carried and held by patient B.

Furthermore, driving unit 3 of the phototherapy device includes main body 301 having power supply switch 305, plug 302, power supply cord 304, and the like. Plug 302 is provided at the tip end of signal cord 303 extending from a part of main body 301, and constitutes fourth coupling portion 6b that is a part of second coupling mechanism unit 6. Furthermore, plug 302 is coupled to socket 204 that is third coupling portion 6a of treatment unit 2. Power supply switch 305 is connected to a commercial power supply, and supplies driving unit 3 with energy. Power supply switch 305 controls an operation of driving unit 3.

Specifically, power supply cord 304 is connected to a commercial power supply. Then, plug 302 of signal cord 303 is set to socket 204 of treatment unit 2. In this state, power supply switch 305 is turned on. Thus, energy from the commercial power supply is supplied to treatment unit 2 via driving unit 3.

In this case, in this exemplary embodiment, second coupling mechanism unit 6 includes socket 204 that is third coupling portion 6a and signal cord 303 provided with plug 302 that is fourth coupling portion 6b.

Furthermore, in this exemplary embodiment, third different-shaped portion 206 constituting a third identifying portion is formed in a part of socket 204, and fourth different-shaped portion 306 constituting a fourth identifying portion that can be fitted into third different-shaped portion 206 is formed in a part of plug 302. Thus, treatment unit 2 and driving unit 3 can identify each other. That is to say, when third different-shaped portion 206 of treatment unit 2 and fourth different-shaped portion 306 of driving unit 3 are fitted into each other, energy is supplied to treatment unit 2 from driving unit 3.

Next, a configuration of an electric circuit of the phototherapy device of the medical device of this exemplary embodiment is described with reference to FIG. 3.

FIG. 3 is a schematic diagram of an electric circuit of the phototherapy device as an example of the medical device.

As shown in FIG. 3, in the phototherapy device as the medical device of this exemplary embodiment, treatment portion 2d includes light-emitting portion 202, and power supply unit 2b includes a power supply circuit portion including, for example, main capacitor 208. Note here that light-emitting portion 202 uses, for example, xenon flash discharge tube 209. Main capacitor 208 is charged via driving unit 3. Furthermore, charging energy of main capacitor 208 is supplied to xenon flash discharge tube 209 so as to allow xenon flash discharge tube 209 to emit light.

That is to say, the charging energy of main capacitor 208 is supplied from driving unit 3. Specifically, power supply switch 305 is turned on in a state in which driving unit 3 is connected to a commercial power supply via power supply cord 304. Then, AC-DC converter circuit 307 converts alternating current energy of the commercial power supply into direct current energy. At the same time, main capacitor 208 is charged by an operation of AC-DC converter circuit 307.

Furthermore, driving unit 3, when it is coupled to treatment unit 2, includes control section 308 connected to a drive control unit including, for example, control circuit 212, of control section 2c. Control section 308 is connected to AC-DC converter circuit 307. At this time, control circuit 212 of treatment unit 2 generates a control signal based on the prescribing information stored in storage unit 2a. Then, control section 308 of driving unit 3 sets a voltage value, which has been converted by AC-DC converter circuit 307, at AC-DC converter circuit 307. Thus, for example, a charging voltage of the main capacitor 208 constituting the power supply circuit portion is set.

Furthermore, xenon flash discharge tube 209 as light-emitting portion 202 is connected to both ends of main capacitor 208 via control element 210 formed of, for example, IGBT (Insulated Gate Bipolar Transistor). Then, an ON/OFF operation of control element 210 is controlled so as to control a light-emitting operation including an amount of light emission, a number of times of light emission, and light emission frequency of xenon flash discharge tube 209. At this time, in this exemplary embodiment, an operation of control element 210 of treatment unit 2 is controlled by control circuit 212. In addition, an operation of control circuit 212 of treatment unit 2 is controlled by storage unit 2a. Specifically, control circuit 212 of treatment unit 2 controls the light-emitting operation of xenon flash discharge tube 209 depending on the prescribing information of a patient stored in storage unit 2a. Note here that starting of an operation of exciting xenon flash discharge tube 209 of treatment unit 2 to emit light is carried out by so-called trigger circuit 211 controlled by control circuit 212.

Hereinafter, an operation of the phototherapy device as the medical device in accordance with the present invention is described with reference to FIGS. 2 and 3. Hereinafter, an example in which a doctor or a medical institution examines a visiting patient and prescribes home treatment using a phototherapy device is specifically described.

In this case, the doctor or the medical institution couples plug 102 that is first coupling portion 5a of first coupling mechanism unit 5 of input unit 1 and socket 203 that is second coupling portion 5b of treatment unit 2 to each other. Then, the doctor or the medical institution inputs and stores prescribing information by the phototherapy device in storage unit 2a of treatment unit 2 based on the result of examination to the patient.

Specifically, the doctor or the medical institution inputs the prescribing information by the phototherapy device into treatment unit 2 via first coupling mechanism unit 5 by manipulating input portion 4 of input unit 1 including keyboard 401, input screen 402, or the like, based on the result of examination to the patient. Then, the input prescribing information is input and stored to be retained in storage unit 2a in treatment unit 2.

On the other hand, after the patient is examined by the doctor or the medical institution, the patient receives treatment unit 2 in which the prescribing information of the patient himself/herself has been input and stored, and driving unit 3 for operating treatment unit 2, as a prescription result, similar to a prescribed drug or the like. Then, the patient takes home the received treatment unit 2 and driving unit 3.

Next, the patient carries out treatment at a patient's home by the following procedure by using treatment unit 2 and driving unit 3 which the patient takes home.

Firstly, the patient couples and connects treatment unit 2 and driving unit 3 to each other. Specifically, the patient couples socket 204 that is third coupling portion 6a constituting second coupling mechanism unit 6 of treatment unit 2 and plug 302 provided at the tip end of signal cord 303 that is fourth coupling portion 6b of driving unit 3 to each other. At this time, for example, based on the fitted state between third different-shaped portion 206 that is a third identifying portion of socket 204 of treatment unit 2 and fourth different-shaped portion 306 that is a fourth identifying portion of plug 302 of driving unit 3, identification between treatment unit 2 and driving unit 3 is carried out. As a result, it is possible to prevent a patient's affected part from being treated by different treatment unit 2.

Next, power supply cord 304 of driving unit 3 is connected to a commercial power supply. Then, power supply switch 305 provided to main body 301 of driving unit 3 is turned on (ON). Thus, an operation of driving unit 3 is started. That is to say, as described with reference to FIG. 3, an operation of AC-DC converter circuit 307 of driving unit 3 is started. Then, main capacitor 208 in treatment unit 2 is charged through AC-DC converter circuit 307 at a voltage value set by control section 308.

Next, at a stage at which charging of main capacitor 208 of treatment unit 2 is completed, light-emitting portion 202 of treatment unit 2 is set toward a site to be treated of the patient. In this state, starting switch 207 of control section 2c of treatment unit 2 is turned on. Thus, control circuit 212 controls a light-emitting operation of xenon flash discharge tube 209 based on prescribing information specific to the patient, which is based on the examination by the doctor and is stored in storage unit 2a. That is to say, control circuit 212 controls operations of control element 210 and trigger circuit 211. Verification of completion of charging of main capacitor 208 of treatment unit 2 is not shown, by is carried out by monitoring a charging voltage of, for example, main capacitor 208. Specifically, various known notifying devices can be used, for example, in a method for notifying by lighting a charge-completion lamp when the charging voltage of main capacitor 208 becomes a predetermined value. This enables a patient to understand the starting state of treatment in treatment unit 2.

Next, when trigger circuit 211 is operated by control circuit 212 of treatment unit 2, the light-emitting operation of xenon flash discharge tube 209 is started. At this time, control circuit 212 controls the ON/OFF operation of control element 210. Thus, light whose amount of light emission, number of times of light emission, or the like, is controlled based on the prescribing information of the patient is output from xenon flash discharge tube 209, and supplied to the treatment site of the patient.

That is to say, as mentioned above, a state in which light output through the light-emitting operation based on the prescribing information is supplied to the treatment site corresponds to a state in which treatment is carried out by the phototherapy device as the medical device of this exemplary embodiment. As a result, a patient can carry out treatment by himself/herself based on the prescribing information by the doctor or the medical institution at a predetermined place such as a patient home.

Note here that in this exemplary embodiment, treatment details, for example, an amount of light emission, a number of times of light emission, or the like, of xenon flash discharge tube 209, which are set by operation control of control element 210 by control circuit 212, are limited by individual properties of main capacitor 208, xenon flash discharge tube 209, or the like. On the other hand, the above-mentioned limitation corresponds to the limitation of the prescription details prescribed by the doctor or the medical institution.

Then, in the phototherapy device as the medical device of this exemplary embodiment, it is preferable to prepare treatment units having different individual properties such as a capacity value of the above-mentioned main capacitor 208 and durability of xenon flash discharge tube 209. Specifically, two or more types of individual treatment units described as treatment units 2A and 2B shown in FIG. 1 are provided. Thus, the above-mentioned limitation by the treatment details is largely relieved, and an affected part of a patient can be treated based on the plurality of treatment details.

That is to say, the doctor or the medical institution selects a treatment unit suitable for prescription details to be prescribed for the patient from the group of some treatment units having different capacities of main capacitor 208 based on the examination of the patient. Then, in the selected treatment unit, operation control of control element 210 by control circuit 212 is set based on the prescription details. Thus, the prescription details for the patient can be made to be more detailed treatment details. As a result, more accurate treatment can be provided to the patient.

Note here that, needless to say, the present invention is not necessarily limited to the above-described embodiment, and any modifications may be made thereto without departing from the gist of the invention.

For example, in FIG. 3, an example in which power supply cord 304 and AC-DC converter circuit 307 are used as a configuration of driving unit 3 is described, but the configuration is not limited to this example. For example, driving unit 3 may be formed of, for example, a battery.

Furthermore, in this exemplary embodiment, the medical device is described taking a phototherapy device as an example. However, the medical device is not limited to this, and may be, for example, a rough-skin treatment device or a rough-skin preventive device. Note here that the rough-skin treatment device or the rough-skin preventive device are devices to be used for preventive treatment mainly for preventing being affected with inflammatory disease, or for reducing symptoms at the time of affection of the disease. That is to say, the rough-skin treatment device and the rough-skin preventive device are devices for applying light onto an affected part of a subject to be treated (a patient), who undergoes treatment of inflammatory disease in order to suppress the inflammatory disease. In this case, the rough-skin treatment device or the rough-skin preventive device include a light-emitting portion with which radiated light having a wavelength of 566.5 nm or more and 780 nm or less is applied.

Specifically, when treatment unit 2 is used for the rough-skin treatment device and the rough-skin preventive device, light-emitting portion 202 of treatment unit 2 emits light in optimum conditions according to treatment conditions such as a site to be treated (for example, shoulder, waist, hand, leg/foot, the whole body, and the like), an irradiation amount (light amount) per treatment, irradiation timing, and an irradiation period. In this case, usually, treatment unit 2 covering all of the above-mentioned treatment conditions need to include main capacitor 208 having capacity capable of corresponding to all the treatment conditions, and xenon flash discharge tube 209 having a size capable of corresponding to all the treatment conditions. However, when treatment unit 2 capable of corresponding to all the treatment is used for all subjects to be treated uniformly regardless of the severity of treatment, a patient having light severity need to carry unnecessarily large and heavy treatment unit 2.

Then, in this exemplary embodiment, as treatment unit 2, two or more types of treatment units 2 whose capacity of main capacitor 208 and size of xenon flash discharge tube 209 are different according to the treatment conditions are prepared. Then, among the two or more types of treatment units 2 having different weight and size, appropriate treatment unit 2 according to the treatment conditions of a patient is used. Thus, it is possible to provide a medical device in which a burden of a patient in carrying treatment unit 2 is reduced, and which is excellent in portability.

Furthermore, the medical device of this exemplary embodiment may include, for example, a low-frequency therapy device. That is to say, treatment unit 2 may include a low-frequency outputting portion of the low-frequency therapy device as treatment portion 2d. This enables a range of treatment details for a patient to be further extended.

Furthermore, the phototherapy device as the medical device of this exemplary embodiment may be provided with, for example, a notifying portion for notifying a lifetime of main capacitor 208 or xenon flash discharge tube 209. The notifying portion may include, for example, a liquid crystal display (for example, displaying a letter “lifetime”) formed on the surface of treatment unit 2, and a light-emitting diode (notifying a lifetime by flickering). In this case, firstly, control circuit 212 monitors charging capacity (a charging voltage) of main capacitor 208 or a light-emitting voltage of xenon flash discharge tube 209. Then, when control circuit 212 senses predetermined charging capacity (for example, reduction of a predetermined amount of charging capacity with respect to the rated charging capacity of main capacitor 208) or a light-emitting voltage (for example, an increase of a predetermined amount of light-emitting voltage of xenon flash discharge tube 209), notifying is carried out via the notifying portion. Thus, it is possible to detect a lifetime of treatment unit 2, and notify a user of the time when treatment unit 2 needs to be replaced by another.

As described above, the medical device of the present invention includes an input unit for inputting prescribing information prescribed by a doctor for an examined patient; a treatment unit for carrying out a treatment operation based on the prescribing information input by the input unit; and a driving unit mounted to the treatment unit and controlling the treatment unit to be in an operable state. The input unit, the treatment unit, and the driving unit are configured to be separable from each other. The treatment unit in which the prescribing information has been input and the driving unit are provided separately for enabling the treatment operation to be carried out at a desired place.

With this configuration, the treatment unit and the driving unit except for the input unit can be set at a desired place such as a patient's home. At this time, for example, when prescription is required to be controlled in more detail, various types of the treatment units themselves are prepared, thus enabling selection corresponding to the detailed prescriptions. Thus, an entire shape of the medical device including the input unit, the selected treatment unit, and the driving unit can be prevented from being increased in size. As a result, home treatment can be achieved while a patient's burden due to cost-up is prevented. Specifically, similar to a case of treatment in which different number of drugs are prescribed depending on symptoms of a patient, various types of treatment units constituting the medical device are prepared in advance. Then, a doctor selects a treatment unit depending on the prescription and provides a patient with the selected treatment unit. Accordingly, it is not necessary to provide a medical device main body or a treatment unit itself with all of the configurations for achieving various types of treatment. As a result, various types of treatment can be achieved by using a medical device having a small size and being excellent in portability.

Furthermore, a prescription determined depending on the conditions of a patient by a doctor or a medical institution can be set to the treatment unit via the input unit, and the treatment unit can be operated via the driving unit at a patient's home. Thus, it is possible to achieve a medical device with which a patient himself/herself can carry out a treatment operation with the prescription determined for the patient under the management of the doctor or the medical institution.

Furthermore, according to the medical device of the present invention, the input unit includes a first coupling portion constituting a first part of a first coupling mechanism unit that couples the input unit to the treatment unit. The treatment unit includes a second coupling portion constituting a second part of the first coupling mechanism unit. The first coupling mechanism unit further includes a first identifying portion of the input unit and a second identifying portion of the treatment unit, which identify each other. The treatment unit may be configured to receive the prescribing information only from the input unit identified.

With this configuration, input of the prescribing information into the treatment unit can be carried out only from the predetermined input unit which is coupled by the first coupling mechanism unit and identified by the first identifying portion. In other words, the input unit can be set at a place where, for example, a doctor or a medical institution can operate. Thus, the input of the prescribing information into the treatment unit is carried out only by the doctor or the medical institution. As a result, it is possible to prevent the input prescribing information from being changed carelessly.

Furthermore, according to the medical device of the present invention, the treatment unit includes a third coupling portion constituting a first part of a second coupling mechanism unit that couples the treatment unit to the driving unit. The driving unit includes a fourth coupling portion constituting a second part of the second coupling mechanism unit. The second coupling mechanism unit further includes a third identifying portion of the treatment unit and a fourth identifying portion of the driving unit, which identify each other. The driving unit may be configured to operate only the identified treatment unit.

With this configuration, the driving unit controls only the operation of the treatment unit coupled by the second coupling mechanism unit and identified by the second identifying portion. That is to say, the treatment unit operates only at a place where the driving unit is set. Thus, a patient can carry out treatment by himself/herself by using the identified treatment unit based on the prescribing information input from the input unit for the patient.

Furthermore, according to the medical device of the present invention, the treatment unit includes two or more types of separable treatment units according to the prescription details. The two or more types of treatment units may have a configuration in which operations are controlled depending on the prescribing information.

With this configuration, selection usually carried out in prescription of drugs, for example, selection of prescription details in which a treatment effect is controlled by the number of prescribed drugs, can be achieved by selection from the two or more types treatment units by a doctor or a medical institution. Furthermore, operation states of the two or more types of treatment units can be also controlled depending on the prescribing information from the input units. Thus, more accurate treatment can be carried out depending on the conditions of an affected part of a patient.

Specifically, firstly, three types of treatment units having different prescription details, for example, treatment units having high, middle, and low treatment effects, are prepared. Then, in the treatment units having high, middle, and low treatment effects, the treatment effect states can further be set to the two or more types, for example, three types. Thus, the doctor or the medical institution can select nine types of treatment prescriptions, as a whole. Therefore, as compared with the case where all of the nine types of treatment prescriptions are achieved by a medical device main body, the size of shape of the medical device can be prevented from being increased and a patient need not bear the cost of configurations unnecessary for treatment prescription of the medical device. As a result, for a patient, a medical device that is excellent in terms of setting space and the cost can be provided.

Furthermore, according to the medical device of the present invention, the treatment unit includes at least a treatment portion for carrying out the treatment operation, a control section for controlling the treatment operation by the treatment portion, a storage unit for storing the prescribing information input from the input unit, and a power supply unit coupled to the driving unit and outputting energy necessary for treatment. Then, the treatment unit may control operation of outputting the energy from the power supply unit and the treatment operation of the treatment portion by the control section based on the prescribing information stored in the storage unit.

With this configuration, treatment based on the prescribing information prescribed by a doctor or a medical institution for a patient can be carried out by using the treatment unit at a place where the driving unit is set. Thus, a medical device for home treatment can be configured. As a result, a patient need not frequently go to a medical institution for undergoing treatment, thus largely reducing a burden in terms of cost.

Furthermore, according to the medical device of the present invention, the power supply unit is controlled to be in a state capable of outputting the energy, which is necessary for the treatment and is supplied from the driving unit coupled via the second coupling mechanism unit. Then, when the treatment unit is separated from the driving unit, the treatment unit may carry out the treatment operation based on the prescribing information that has been input from the input unit.

With this configuration, the energy necessary for treatment is output from the driving unit to the power supply unit of the treatment unit, and accumulated. Thereafter, the treatment unit is separated from the driving unit, and a treatment operation by the treatment portion can be carried out with accumulated energy in a place other than a place at which the power supply unit is set. As a result, the degree of freedom of selection of treatment places can be extremely increased.

Furthermore, according to the medical device of the present invention, the treatment portion of the treatment unit includes a light-emitting portion for emitting therapeutic light. The control section of the treatment unit controls a light-emitting operation of the light-emitting portion. The power supply unit of the treatment unit includes a power supply circuit portion including, for example, a main capacitor, coupled to the driving unit and accumulating light-emitting energy. Then, the power supply unit may be configured to control an operation of accumulating the light-emitting energy by the power supply circuit portion and a light-emitting operation of the control section based on the prescribing information stored in the storage unit.

With this configuration, the light-emitting operation of the light-emitting portion for treatment can be carried out at a place at which the driving unit is set based on the prescribing information prescribed for a patient by the doctor or the medical institution. Thus, the phototherapy device for home treatment can be configured. As a result, a patient need not frequently go to a medical institution for undergoing treatment, thus largely reducing the burden in terms of cost.

Furthermore, according to the medical device of the present invention, the treatment unit further includes a drive control unit controlling operation of the driving unit based on the prescribing information stored in the storage unit. The driving unit may be configured to control light-emitting energy accumulated in the power supply circuit portion based on the prescribing information.

With this configuration, an amount of light-emitting energy accumulated in the power supply circuit portion can be controlled by the driving unit. Thus, as compared with the case where the amount of light-emitting energy accumulated in the power supply circuit portion itself is controlled, the size of the power supply circuit portion can be reduced, resulting in downsizing the treatment unit itself and simplifying the configuration.

### INDUSTRIAL APPLICABILITY

The present invention can be applied to a medical device with which a treatment operation with a prescription depending on a patient is required to be carried out by the patient himself/herself at a predetermined place under management of the doctor or the medical institution. Thus, home treatment can be achieved without increasing a burden of a patient.

### REFERENCE MARKS IN THE DRAWINGS

1 input unit
2, 2A, 2B treatment unit
2a storage unit
2b power supply unit
2c control section
2d treatment portion
3 driving unit
4 input portion
5 first coupling mechanism unit
5a first coupling portion
5b second coupling portion
6 second coupling mechanism unit
6a third coupling portion
6b fourth coupling portion
101 main body
102 plug
103 first different-shaped portion (first identifying portion)
201, 301 main body
202 light-emitting portion
203, 204 socket
205 second different-shaped portion (second identifying portion)
206 third different-shaped portion (third identifying portion)
207 starting switch
208 main capacitor (power supply circuit portion)
209 xenon flash discharge tube
210 control element
211 trigger circuit
212 control circuit (drive control unit)
302 plug
303 signal cord
304 power supply cord
305 power supply switch
306 fourth different-shaped portion (fourth identifying portion)
307 AC-DC converter circuit
308 control section
401 keyboard
402 input screen

1. A medical device comprising:
an input unit for inputting prescribing information prescribed by a doctor for an examined patient;
a treatment unit for carrying out a treatment operation based on the prescribing information input by the input unit; and
a driving unit mounted to the treatment unit and controlling the treatment unit to be in an operable state,
wherein the input unit, the treatment unit, and the driving unit are configured to be separable from each other, and
the treatment unit in which the prescribing information has been input and the driving unit are provided separately for enabling the treatment operation to be carried out at a desired place.

2. The medical device of claim 1, wherein:
the input unit includes a first coupling portion constituting a first part of a first coupling mechanism unit that couples the input unit to the treatment unit;
the treatment unit includes a second coupling portion constituting a second part of the first coupling mechanism unit;
the first coupling mechanism unit further includes a first identifying portion of the input unit and a second identifying portion of the treatment unit, which identify each other; and
the treatment unit receives the prescribing information only from the input unit identified.

3. The medical device of claim 1, wherein:
the treatment unit includes a third coupling portion constituting a first part of a second coupling mechanism unit that couples the treatment unit to the driving unit;
the driving unit includes a fourth coupling portion constituting a second part of the second coupling mechanism unit;
the second coupling mechanism unit further includes a third identifying portion of the treatment unit and a fourth identifying portion of the driving unit, which identify each other; and
the driving unit operates only the treatment unit identified.

4. The medical device of claim 1, wherein the treatment unit includes two or more types of separable treatment units depending on prescription details, and
in each of the two or more types of the treatment units, an operation is controlled depending on the prescribing information.

5. The medical device of claim 1,
wherein the treatment unit includes at least a treatment portion for carrying out the treatment operation, a control section for controlling the treatment operation by the treatment portion, a storage unit for storing the prescribing information input from the input unit, and a power supply unit coupled to the driving unit and outputting energy necessary for treatment, and
the treatment unit controls operation of outputting the energy from the power supply unit and the treatment operation of the treatment portion by the control section based on the prescribing information stored in the storage unit.

6. The medical device of claim 5, wherein the power supply unit is controlled to maintain a state capable of outputting the energy, which is supplied from the driving unit coupled via the second coupling mechanism unit, and is necessary for the treatment, to the treatment unit, and
when the treatment unit is separated from the driving unit, the treatment unit carries out the treatment operation based on the prescribing information that has been input from the input unit.

7. The medical device of claim 5, wherein
the treatment portion of the treatment unit includes a light-emitting portion for emitting therapeutic light,
the control section of the treatment unit controls a light-emitting operation of the light-emitting portion,
the power supply unit of the treatment unit includes a power supply circuit portion coupled to the driving unit and accumulating light-emitting energy, and controls operation of accumulating the light-emitting energy by the power supply circuit portion and the light-emitting operation of the light-emitting portion by the control section based on the prescribing information stored in the storage unit.

8. The medical device of claim 7, wherein
the treatment unit further comprises a drive control unit for controlling operation of the driving unit based on the prescribing information stored in the storage unit, and
the driving unit controls light-emitting energy accumulated in the power supply circuit portion based on the prescribing information.
